(19) United States
(12) Patent Application Publication
(10) Pub. No.: US 2005/0267398 A1
(43) Pub. Date: Dec. 1, 2005

(54) Glaucoma shunt

(76) Inventors: Dimitri Protopsaltis, Memphis, TN (US); Anthony D. Prescott, Arlington, TN (US); Edward C. Lindsey JR., Memphis, TN (US)

Correspondence Address: Gordon & Jacobson, P.C., Suite 407, 60 Long Ridge Road, Stamford, CT, 06902, US

(21) Appl. No.: 11/137,044

(22) Filed: May 25, 2005

Related U.S. Application Data
(60) Provisional application No. 60/574,781, filed on May 27, 2004.

(51) Int. Cl. A61M 5/00

(52) U.S. Cl. 604/8

(57) ABSTRACT

A glaucoma shunt includes a plate having filtration and fixation portions. In some embodiments the filtration portion includes a surface provided with stiffening ribs which increase rigidity and an upper surface provided with a ridge. The fixation portion extends anteriorly to facilitate access thereto. A drainage tube is recessed within the plate and extends through the ridge to a central portion of the filtration portion. By emptying the tube at the central portion of the filtration portion, the tube outlet is distanced from perimetric scar tissue which can otherwise obstruct the tube outlet. The ridge keeps tissue over the plate spaced from the outlet of the tube to further facilitate drainage.

### BACKGROUND OF THE INVENTION

1. Field of the Invention

This invention relates broadly to ocular implants. More particularly, this invention relates to ocular implants for transporting aqueous and used in the treatment of glaucoma.

2. State of the Art

Intraocular pressure in the eye is maintained by the formation and drainage of the aqueous. Aqueous is a clear, colorless fluid that fills the anterior and posterior chambers of the eye. Aqueous is a product of the ciliary body in the eye and is a carrier of nutrients for the lens. In addition, aqueous provides a continuous stream into which surrounding tissues can discharge the waste products of metabolism.

Aqueous produced in the ciliary body circulates from the posterior chamber to the anterior chamber of the eye through the pupil and is absorbed through the trabecular meshwork, a plurality of crisscrossing collagen cords covered by endothelium. Once through the trabecular meshwork, aqueous passes through Schlemm's canal and into venous circulation. The rate of aqueous outflow through the trabecular meshwork in a normal eye is typically 2 to 5 μL/min.

Glaucoma is a progressive disease of the eye characterized by a gradual increase of intraocular pressure. This increase in pressure is most commonly caused by stenosis or blockage of aqueous outflow, resulting in excessive buildup of aqueous fluid in the chambers of the eye. Other causes include increase in venous pressure outside the eye which is reflected back through the aqueous drainage channels and increased production of aqueous. This increase in intraocular pressure produces gradual and permanent damage to the optic nerve resulting in loss of vision in the afflicted eye.

Existing corrective methods for the treatment of glaucoma include drugs, non-implant surgery, and implant surgery. The most common type of implant is a shunt generally including a single layer plate and a draining tube. The plate is sutured onto the sclera of the eye between the rectus muscles, and the drainage tube includes a first end coupled to a periphery of the plate and a second end implanted into the anterior chamber of the eye through a scleral incision adjacent the limbus. The first end of the drainage tube may be open or provided with a valve to control release of aqueous through the tube. By way of example, U.S. Pat. No. 5,454,796 to Krupin; U.S. Pat. Nos. 5,178,604, 5,397,300, 5,476,445, 5,558,629, and 6,050,970 to Baerveldt; U.S. Pat. Nos. 5,071,408, 5,411,473, 5,681,275, 5,743,869, 5,785,675, and 6,261,256 to Ahmed; and U.S. Pat. No. 4,750,901 to Molteno disclose implants as broadly discussed above. Once implanted, a scar tissue bleb forms around the plate. After bleb formation, the bleb controls the release and flow rate of aqueous transported by the tube and, if successful, regulates and normalizes the pressure within the eye. A large bleb is desirable as it filters a greater volume of aqueous, provided however the device which initiates bleb formation should not impinge on the rectus muscles or optic nerve.

In addition, prior art single plate shunt devices (as opposed to devices which include multi-layer overlying plates) terminate the drainage tube at the perimeter of the plate. When scar tissue bleb formation occurs, the bleb about the plate of such a device may obstruct or block the outflow of aqueous through the drainage tube. Such will prevent desirable results for the treatment by failing to regulate intraocular pressure to desirable levels.

### SUMMARY OF THE INVENTION

It is an object of the invention to provide a glaucoma shunt for the eye which creates a large bleb for better filtration and increased outflow of aqueous.

It is another object of the invention to provide a glaucoma shunt for the eye which is easier for the surgeon to implant and requires a shorter procedure for implantation.

It is a further object of the invention to provide a glaucoma shunt which has a drainage tube having an outlet adjacent the plate which will not be blocked by bleb formation.

It is also an object of the invention to provide a glaucoma shunt which has a shape designed to elicit minimal undesired foreign body response by the tissues of the eye.

It is an additional object of the invention to provide a glaucoma shunt which has a reduced profile and is very thin, but has relatively high rigidity.

In accord with these objects, which will be discussed in detail below, a glaucoma shunt is provided which includes a flexible, polymeric filtration plate and a flexible drainage tube. The plate includes filtration and fixation portions. The filtration portion includes upper and lower surfaces, a large posterior portion with a concave rear edge providing additional clearance for the optic nerve of the eye, and a relatively narrower anterior portion. The lower surface is substantially spherically concave to contour to the surface of the sclera, and is provided with stiffening ribs which increase rigidity of the thin flexible plate and further space the plate relative to the tissue to provide better filtration. The filtration portion is sized to have the maximum possible surface area positionable on the sclera within a quadrant of the eye which does not impinge on the rectus muscles and which does not interfere with the optic nerve.

The fixation portion of the plate is integral with the anterior portion and extends anteriorly relative to the prior art to facilitate access thereto for stitching the fixation portion to the sclera. A narrow waist is defined between the filtration and fixation portions. The fixation portion includes a front edge, an opening in the front edge, and an upper channel continuous therewith and extending into a central portion of the plate. The fixation portion optionally includes reference holes for suture placement.

A ridge is provided on the upper side of the plate, over an intermediate portion of the channel. The drainage tube extends through the opening in the front channel and is at last partially recessed within the channel so as to have a low profile relative to the plate. The tube has a first end terminating within the channel at a central portion of the plate, preferably beyond the ridge but spaced from the end of the channel. The ridge lifts the eye tissue off the first end of the tube to prevent obstruction of the first end of the tube. By locating the first end of the tube centrally, it is distanced from perimetric scarring which can result in tube outlet blockage. The first end may be valved or non-valved. The tube has sufficient length such that the second end may be inserted through an incision in the sclera adjacent the limbus and implanted within the anterior chamber of the eye.

In another embodiment of the invention, the plate of the implant is provided with lateral extensions of posterior and anterior portions of the plate. In addition, the embodiments of the implant according to the invention may be optionally provided with fenestration holes to enhance filtration.

Additional objects and advantages of the invention will become apparent to those skilled in the art upon reference to the detailed description taken in conjunction with the provided figures.

### BRIEF DESCRIPTION OF THE DRAWINGS

FIG. 1 is a bottom perspective view according to a first embodiment of a glaucoma shunt according to the invention;

FIG. 2 is a top perspective view according to the first embodiment of a glaucoma shunt according to the invention;

FIG. 3 is a side elevation view according to the first embodiment of a glaucoma shunt according to the invention;

FIG. 4 is a second embodiment of a glaucoma shunt according to the invention;

FIG. 5 is a third embodiment of a glaucoma shunt according to the invention;

FIG. 6 is a schematic illustration of a glaucoma shunt of the invention shown implanted on a eye in accord with the invention;

FIG. 7 is a posterior top perspective view of a plate of a fourth embodiment of a glaucoma shunt according to the invention;

FIG. 8 is an anterior top perspective view of the plate of the fourth embodiment of the glaucoma shunt shown in FIG. 7;

FIG. 9 is an anterior bottom perspective view of the plate of the fourth embodiment of the glaucoma shunt shown in FIG. 7;

FIG. 10 is an anterior elevation of a fifth embodiment of a glaucoma shunt according to the invention;

FIG. 11 is an anterior top perspective view of the fifth embodiment of the glaucoma shunt of FIG. 10;

FIG. 12 is a bottom perspective view of the fifth embodiment of the glaucoma shunt of FIG. 10;

FIG. 13 is an enlarged view of a channel defined by crossbeams in the embodiment shown in FIG. 12;

FIG. 14 is a bottom perspective view of the fifth embodiment of the glaucoma shunt of FIG. 10, shown with a cover of the channel shown in FIGS. 12 and 13;

FIG. 15 is a bottom perspective view of a plate of a sixth embodiment of the glaucoma shunt according to the invention;

FIG. 16 is an anterior top perspective view of the plate shown in FIG. 15;

FIG. 17 is an anterior elevation view of a seventh embodiment of a plate of a glaucoma shunt according to the invention;

FIG. 18 is a top posterior perspective view of the plate of the shunt of FIG. 18;

FIG. 19 is an anterior perspective view of the plate of the shunt of FIG. 18;

FIG. 20 is a bottom perspective view of the plate of the shunt of FIG. 18;

FIG. 21 is another top perspective view of the plate of the shunt of FIG. 18;

FIG. 22 is an anterior top perspective view of a plate of a ninth embodiment of a glaucoma shunt according to the invention;

FIG. 23 is a posterior perspective view of the shunt of FIG. 22;

FIG. 24 is a bottom perspective view of the shunt of FIG. 22; and

FIG. 25 is a top view of a tenth embodiment of a glaucoma shunt according to the invention.

### DETAILED DESCRIPTION OF THE PREFERRED EMBODIMENTS

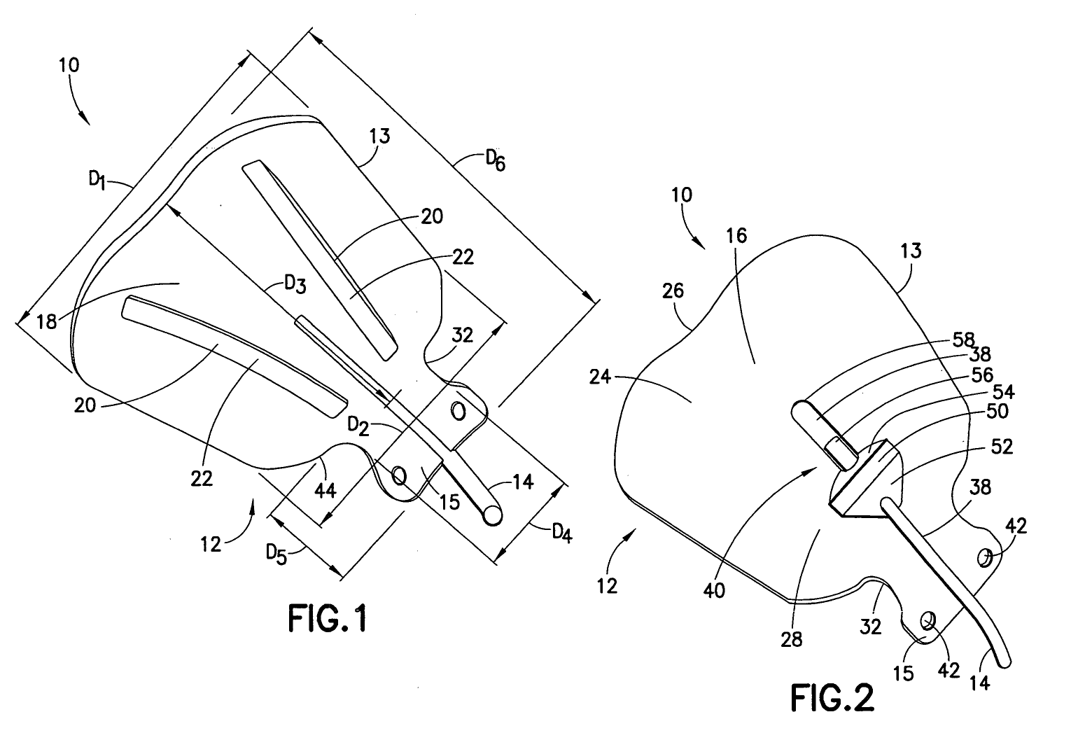

Turning now to FIGS. 1 through 3, a glaucoma shunt 10 for reducing intraocular pressure is shown. The shunt 10 includes a flexible, polymeric filtration plate 12 preferably made of silicone and a flexible drainage tube 14 also preferably made of silicone.

The plate 12 includes a filtration portion 13, about which a bleb forms after implantation, and a fixation portion 15, utilized to secure the shunt to the sclera and about which bleb formation generally does not occur. The filtration portion 13 includes an upper surface 16 and a lower surface 18. The lower surface 18 is substantially spherically concave to contour to the surface of the sclera. In addition, the lower surface 18 is provided with stiffening ribs 20 preferably extending substantially the majority of the anterior-posterior length of the plate which increase rigidity of the thin flexible plate and further space the plate relative to the tissue to provide better filtration. The ribs 20 preferably have a flat lower surface 22. The filtration portion 13 of the plate 12 includes a large proximal portion 24 with a concave rear edge 26 providing additional clearance for the optic nerve of the eye, and tapers to a relatively narrower anterior portion 28.

The fixation portion 15 is integral with the anterior portion 28 of the filtration portion 13 of the plate 12, and extends anteriorly to facilitate access thereto for stitching the fixation portion to the sclera. The fixation portion defines a narrow waist 32 at the junctions of the filtration and fixation portions 13, 15. After implantation, a bleb forms over filtration portion 13 of the plate 12 and crosses the plate 12 at the waist 32 (i.e., does not form about the fixation portion 15). Thus, the size of the bleb is defined by the area of the filtration portion 13. The filtration portion 13 of the plate 12, as further discussed below, is sized to have the maximum possible surface area positionable on the sclera within a quadrant of the eye which does not impinge on the rectus muscles and which does not interfere with the optic nerve.

The fixation portion 15 includes a front edge 34, an opening 36 in the front edge 34, and an upper channel 38 continuous therewith and extending into a central portion 40 of the plate 12. The fixation portion 15 optionally includes reference holes 42 for suture placement. However, the silicone material of the plate 12 is soft enough to permit suturing through any location of the fixation portion 15, and the reference holes 42 are providing only as guides.

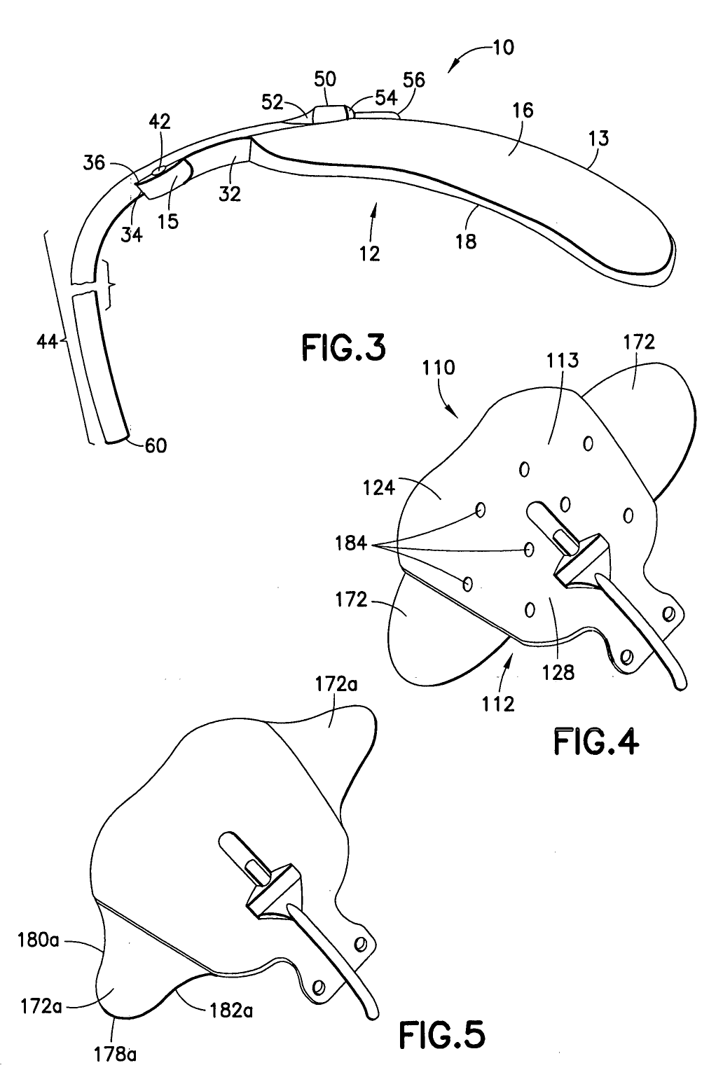

Referring to FIGS. 2 and 3, a ridge 50 is provided on the upper side 16 of the filtration portion 13 of the plate 12, over an intermediate portion of the channel 38. The ridge 50 preferably has sloped or curved anterior and posterior walls 52, 54, and is curved in a lateral (transverse) dimension, all to minimize abrupt contour changes that may otherwise trigger a deleterious tissue response to the plate 12. The drainage tube 14 extends through the opening 36 in the front edge 34 and within the channel 38 so as to be at least partially, and preferably substantially, recessed within the fixation plate 15 and filtration plate 13 to have a low profile relative to the plate 12, again to minimize a negative tissue response. The tube 14 has a first end (outlet end) 56 terminating within the channel 38 at the central portion 40 of the filtration plate 13, beyond the ridge 50 but spaced from the end 58 of the channel 38. The ridge 50 lifts the tissue off the first end 56 of the tube 14 to prevent obstruction of the first end of the tube. By locating the first end 56 of the tube 14 centrally relative to the filtration plate 13, it is distanced from perimetric scarring which can result in tube outlet blockage. More particularly, the scar tissue bleb which forms about the perimeter of the filtration portion 13 after implantation could block an outlet of a drainage tube which opens adjacent the perimeter of the plate. By locating the first end 56 substantially centrally, the likelihood of such blockage is greatly reduced. The first end 56 of the tube is optionally provided with a one-way valve, e.g. an X-shaped slit valve, which opens to allow transport and release of aqueous when subject to predetermined anterior chamber pressure. Alternatively, the first end 56 may be open. The tube 14 has sufficient length such that the second end 60 may be cut to a desired length and inserted through an incision in the sclera adjacent the limbus and implanted within the anterior chamber of the eye.

By way of example, and not by limitation, one embodiment of the shunt 10 of the invention has the following dimensions: the width D₁ across the posterior portion 24 of the plate 10 is preferably approximately 16 mm, the width D₂ across the anterior portion 26 of the plate 10 is preferably approximately 12.4 mm, the anterior-posterior length D₃ of the filtration portion 13, from the concavity 26 in the posterior portion 24 to the waist 32 between the filtration and fixation portions 13, 15 is preferably approximately 15.7 mm, the width D₄ across the waist 32 is preferably approximately 4.8 mm, the length D₅ of the fixation portion from the anterior edge 44 of the filtration portion is approximately 4.7 mm, and the overall anterior-posterior arc length D₆ of the plate 12 is preferably approximately 22.2 mm. Such dimensions provide an overall plate surface area of 279 mm², and a 249 mm² surface area for the filtration portion 13 of the plate 12.

By way of example, and not by limitation, another embodiment of the shunt 10 of the invention has the following dimensions: the width D₁ across the posterior portion 24 of the plate 10 is preferably approximately 13 mm, the width D₂ across the anterior portion 26 of the plate 10 is preferably approximately 12 mm, the anterior-posterior length D₃ of the filtration portion 13, from the concavity 26 in the posterior portion 24 to the waist 32 between the filtration and fixation portions 13, 15 is preferably approximately 14 mm, the width D₄ across the waist 32 is preferably approximately 4.8 mm, the length D₅ of the fixation portion from the anterior edge 44 of the filtration portion is approximately 4 mm, and the overall anterior-posterior arc length D₆ of the plate 12 is preferably approximately 18 mm.

Turning now to FIG. 4, another embodiment of a glaucoma shunt implant 110 according to the invention, substantially similar to implant 10, is shown. Shunt 110 includes a pair of lateral extensions 172 of the posterior and anterior portions 124, 128 of the filtration portion 113 of the plate 112. In one embodiment, the lateral extensions 172 have continuously convexly curving outlines. Alternatively, as shown in FIG. 5, the lateral extensions 172a may be defined by a combination of convex 178a and concave curves 180a, 182a. The shunts of FIGS. 4 and 5 with lateral extensions are irregular in shape with dramatic changes in curvature, in distinction from a regular laterally extended shape, e.g., elliptical. Referring back to FIG. 4, any of the described embodiments may be optionally provided with fenestration holes 184 to enhance filtration.

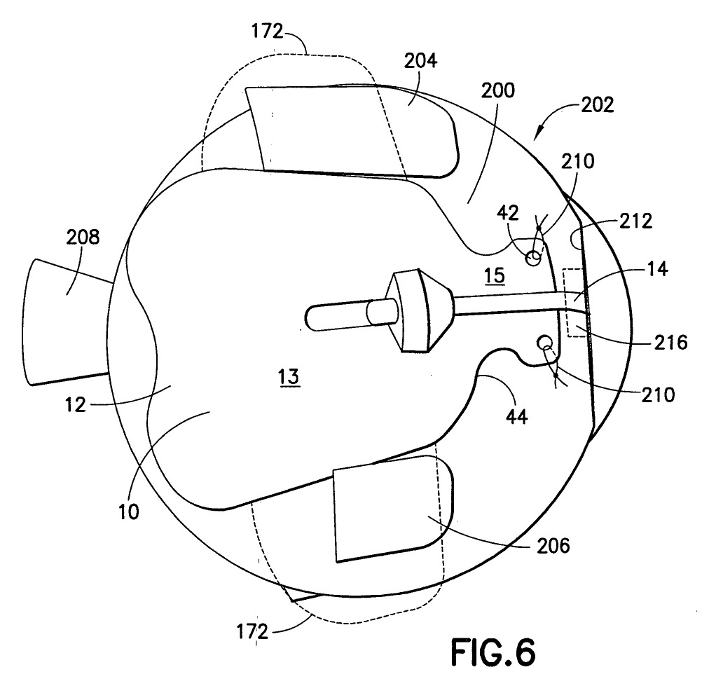

Referring to FIGS. 1 through 3 and 6, during implantation, a 90° to 110° fornix-based or limbal-based incision is made through the conjuctiva and Tenon's capsule into a quadrant of the eye between two rectus muscles 204, 206, and preferably into the superior temporal quadrant. Adequate scleral exposure is obtained so that the anterior edge 44 of the filtration portion 13 of the plate 12 is preferably at least 8-9 mm posterior to the limbus 212. The shunt 10 is positioned within the quadrant, ensuring that there is also adequate posterior clearance relative to the optic nerve 208 (at least 2 mm) and that the sides of the filtration portion 13 do not impinge on the rectus muscles 204, 206. As discussed above, the dimensions of the filtration portion are maximized such that the filtration portion 13 fits within a quadrant of the eye, but does not impinge on the insertion of the rectus muscles and does not interfere with the optic nerve 208. If the shunt 10 includes lateral extensions, such as 172 shown in broken lines, the extensions 172 may extend under the rectus muscles 204, 206 or even over the rectus muscles, as shown. As also discussed above, the ribs 20 provide flexural rigidity to facilitate insertion of the plate 12 between the layers of tissue defined by the incision.

The fixation portion 15 of the plate 12 is then sutured to the sclera 200 with interrupted, nonabsorbent sutures 210. The sutures 210 may be provided through the fixation holes 42 or sewn through any other portion of the fixation portion 15. The relatively anterior position of the fixation portion 15, extending forward preferably approximately 5 mm from the anterior edge 44 of the filtration portion 13, provides easier surgeon access for anchoring the implant, requires that less of the conjunctiva be incised, and permits a faster surgical procedure.

The tube 14 should face the limbus 212. A separate corneal paracentesis tract is made before the limbal entry incision, and while a viscoelastic agent can be injected into the anterior chamber, over inflation of the anterior chamber is avoided. If vitreous is present in the anterior chamber, an automated vitrectomy is performed via a separate entry site, prior to insertion of the tube 14. Two types of limbal entry incisions are possible: full thickness, or preferably within the bed of a 4 mm×4 mm ½ thickness lamellar scleral flap. If a lamellar scleral flap is created, the flap is dissected into clear cornea, improving visualization of the limbal anatomy and allowing more accurate tube placement into the anterior chamber. A full thickness entry tract is indicated if the sclera is extremely thin, making dissection of a flap difficult. Depending upon access and incision, the tube may be cut to a shorter length, if desired. The second end of the tube is inserted into the anterior chamber. A tissue graft 216 is then sutured over the limbal entry incision.

After implantation and prior to bleb formation, a valve, if provided at the first end 56 of the tube 14, provides flow control to prevent hypotony. If the first end 56 is non-valved, it is preferable that dissolvable sutures be provided through the tube 14 which prevent or limit uncontrolled aqueous flow through the drainage tube until bleb formation. After bleb formation (caused by scar tissue formation about the periphery of the filtration portion 13 of the plate 12), regardless of the presence of a valve, the bleb controls the maximum flow rate of the drainage of aqueous from the anterior chamber through the drainage tube 14, onto the plate 12 and into the surrounding tissue.

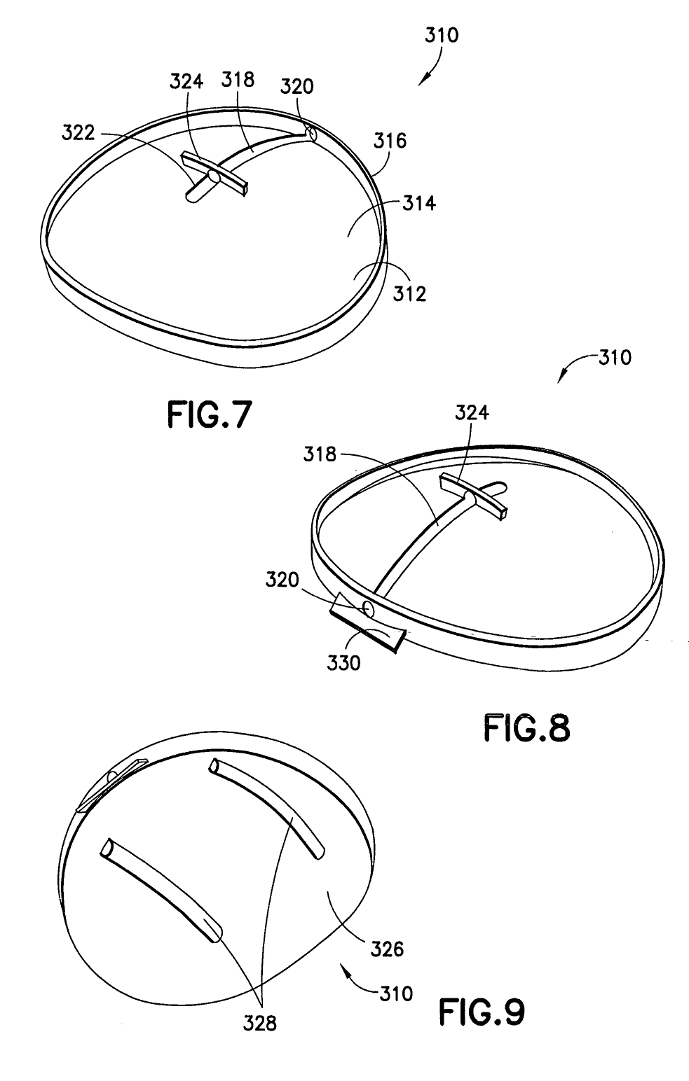

Turning now to FIGS. 7 through 9, another embodiment of a glaucoma shunt implant 310 is shown. Implant 310 includes a generally elliptical elastomeric plate 312 with an upper convex upper surface 314 with a low wall 316 around the perimeter of the plate to lift tissue off the plate at the perimeter thereof. The upper surface 314 also includes a recessed channel 318 for a drainage tube (not shown for clarity but similar to drainage tube 14), and a hole 320 is provided in the wall 316 for the tube. The channel 318 terminates centrally at 322 on the plate 312 so that the tube outlet is distanced from perimetric scarring which could otherwise cause outlet blockage. An anchoring ridge 324 is provided over the channel 318 to secure the end of the tube in the channel and lift eye tissue off the surface 314 of the plate 312 adjacent the tube outlet. The implant 310 includes a lower concave surface 326 provided with ribs 328 which lift the surface 326 relative to the eye tissue and permit fluid filtration. The anterior edge of the implant 310 is provided with a flange 330 by which the implant 310 can be secured to the eye with sutures. The surface area of the plate 312 is preferably approximately at least 350 mm².

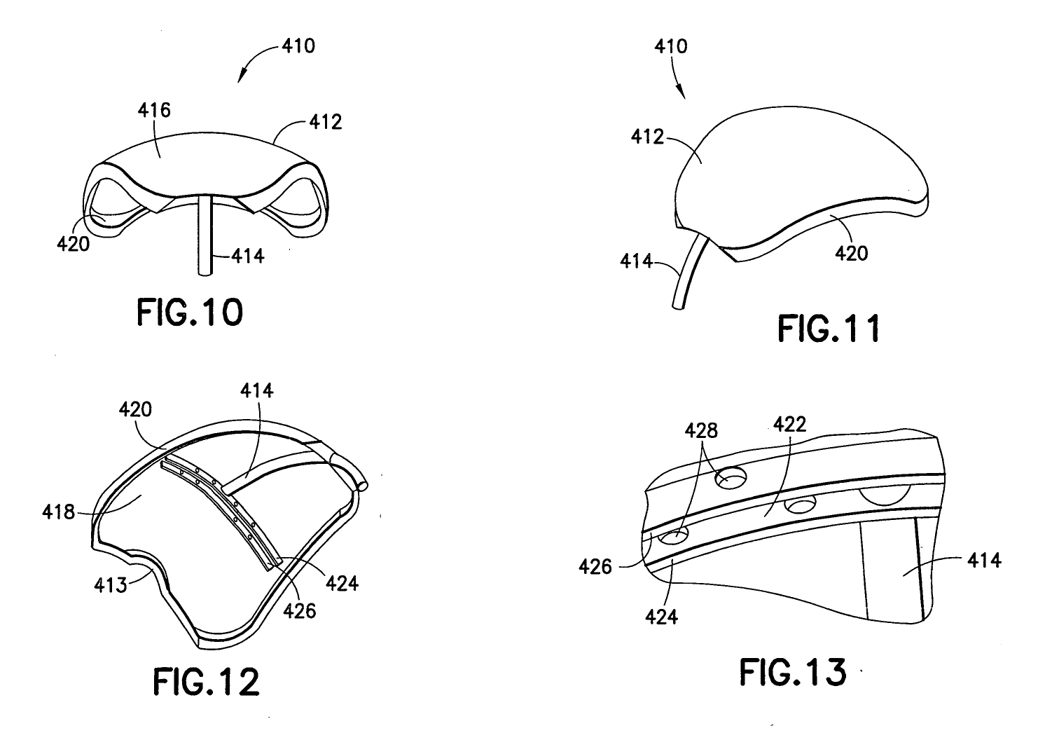

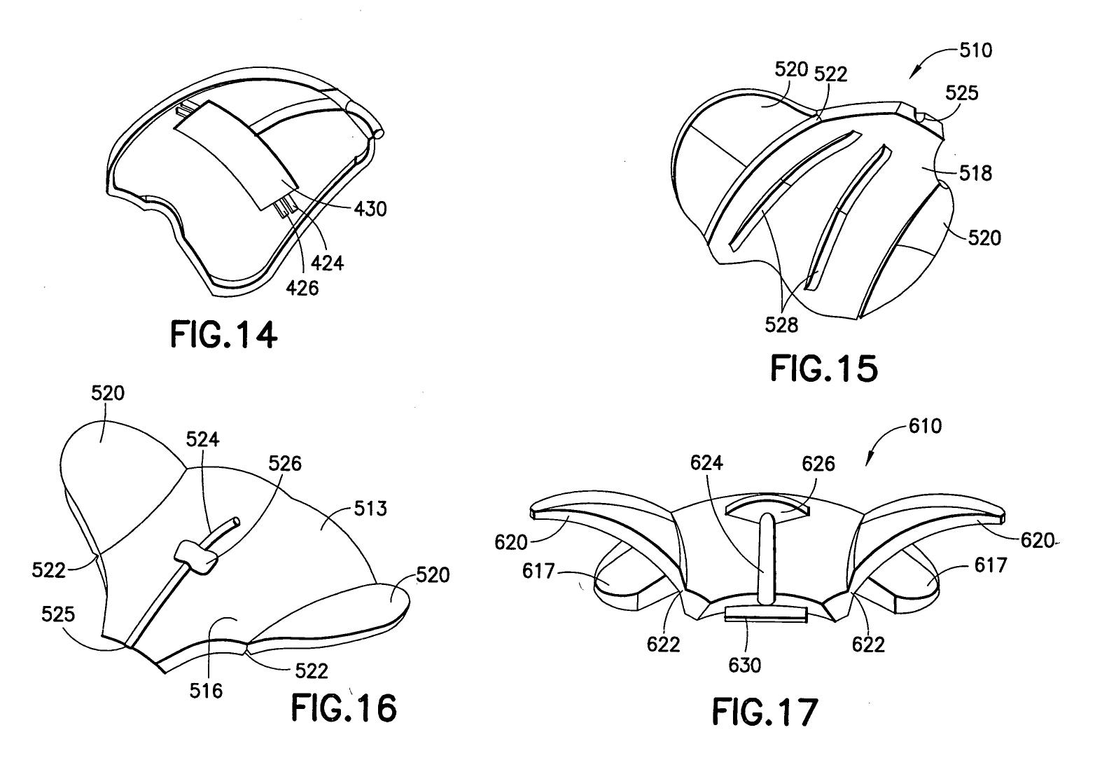

Referring to FIGS. 10 through 14, another embodiment of a glaucoma shunt implant 410 is shown. The implant 410 includes an elastomeric plate 412 and a tube 414. The plate 412 expands in a posterior direction, but is sized to seat between two rectus muscles. A recess 413 is provided at the posterior edge of the plate to accommodate the optic nerve. The plate 412 includes a convex upper surface 416 and a concave lower surface 418 with a peripheral wall 420 descending about the lower surface 418. The wall is adapted to lift the peripheral portion of the lower surface 418 relative to the underlying tissue. The tube 414 extends through a hole in the wall 420 and runs along lower surface 418 to a central location where the outlet of the tube empties into a channel 422 defined by two crossbeams 424, 426 extending laterally across the lower surface 418 of the plate 412. The crossbeams 424, 426 also operate to lift the lower surface 418 relative to the underlying tissue. The crossbeams 424, 426 include perforations (i.e., drain holes) 428 which allow filtration of aqueous therethrough. The perforations 428 on the crossbeams are offset relative to each other to slow or prevent potential blockage from in-growth into the channel 422. A silicone flap 430 is provided over the crossbeams 424, 426 and thus the channel 422 to prevent fibril in growth into the outlet of the tube and the perforations 428 and to provide for diffuse filtration. The crossbeam and silicone flap elements can be incorporated into any of the other above and below described embodiments. The surface area of the plate 412 is preferably approximately at least 350 mm².

Turning now to FIGS. 15 and 16, another embodiment of a glaucoma shunt implant 510 is shown. Implant 510 includes an elastomeric plate 5 12 and a flexible tube (not shown, but similar to prior described tubes). The plate 512 includes a central portion 513 having a convex upper surface 516 and a convex lower surface 518, and laterally extending wing portions 520 which are attached by live hinges 522 to the central portion 513. The live hinges 522 permit wing portions 520 to flex upwards relative to the central portion 513 of the plate 512. As the wing portions can flex upwards, the central portion 513 can be positioned between two rectus muscles, and the wings 520 can be situated over (on top of) the rectus muscles. The hinges 522 allow the wings 520 to move with the rectus muscles without inhibiting movement of the rectus muscles. The upper surface 516 includes a channel 524 with a lowered tube entry 525 which extends to a central location on plate for recessing the tube therein. The lowered tube entry 525 causes reduced tissue erosion. A ridge portion 526 holds down the tube and lifts the tissue off the outlet of the tube. The lower surface 518 includes anterior-posterior ribs 528 which are adapted to lift the lower surface 518 relative to underlying tissue. The surface area of the plate 512 (central portion and wings together) is preferably at least 350 mm². A bleb forms over the entire plate 512.

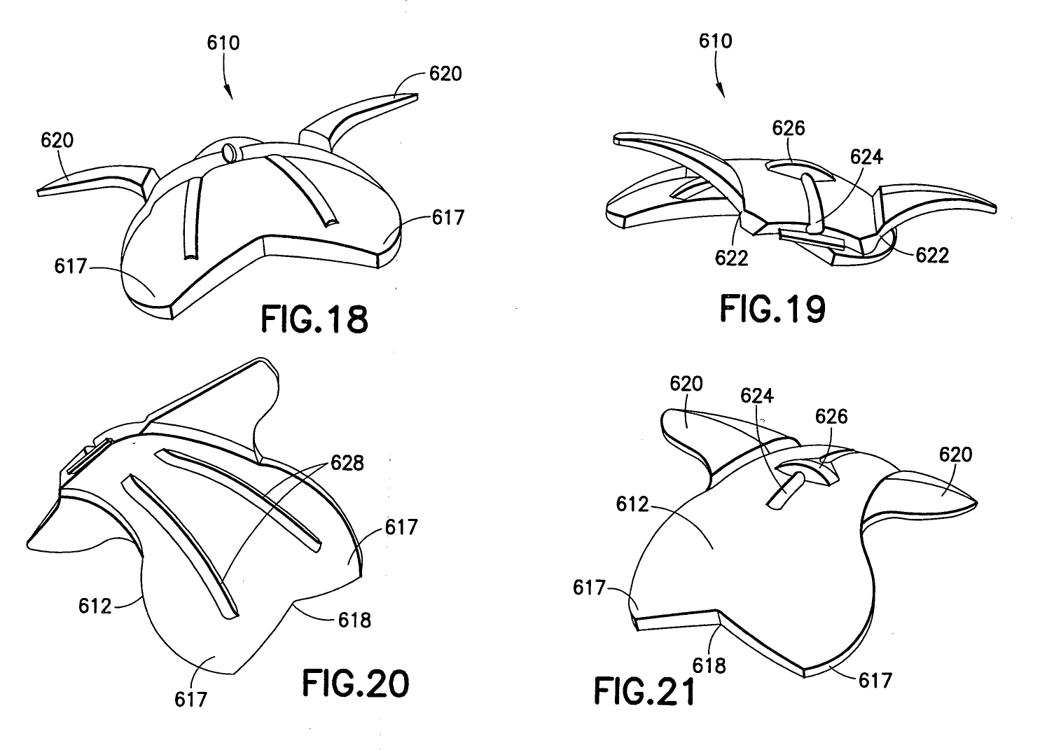

Referring now to FIGS. 17 through 21, another embodiment of a glaucoma shunt implant 610 is shown. The implant 610 includes a plate 612 contoured to fit the surface of the eye and including an anterior portion 614 and a laterally expanding posterior portion 616 defining posterior wings 617 adapted to seat under a pair of rectus muscles. The posterior portion 616 includes notch 618 to accommodate the optic nerve. A pair of anterior gull-type wings 620 are laterally coupled to the anterior portion of the plate by live hinges 622. Similar to prior embodiments, the plate 612 includes a channel 624 for a drainage tube (not shown), and a ridge for lifting tissue relative to the outlet of the tube. Ribs 628 are also provided along an undersurface of the plate 612. An anterior flange 630 is also provided for securing the plate to the eye. The surface area of the plate 512 (plate with anterior and posterior wings) is preferably at least 350 mm². The multiple sets of wings allow for larger bleb formation without requiring substantial manipulation of the anatomy during implantation.

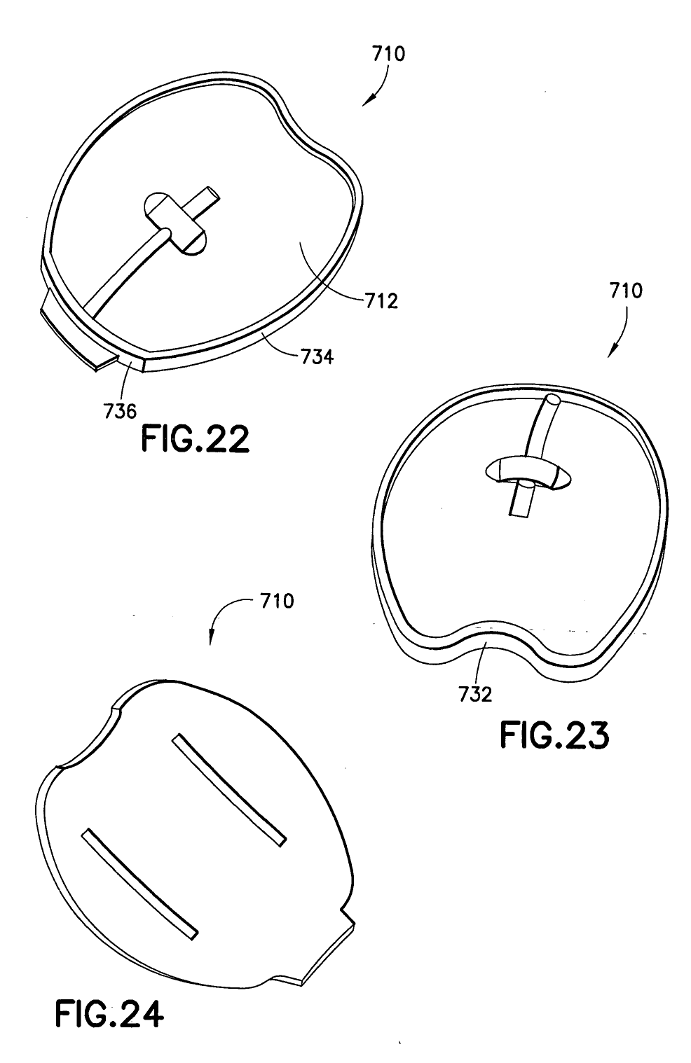

Turning now to FIGS. 22 through 24, another embodiment of a glaucoma shunt implant 710 according to the invention is shown. The implant 710 is substantially similar to the embodiment is shown in FIGS. 7 through 9 generally including all features described with respect thereto, and differs only in shape. In that regard, the plate 712 of the implant 710 has a greater anterior-posterior dimension than lateral dimension, and includes a posterior notch 732 to accommodate the optic nerve. The surface area of the plate 312 is preferably approximately 350 mm².

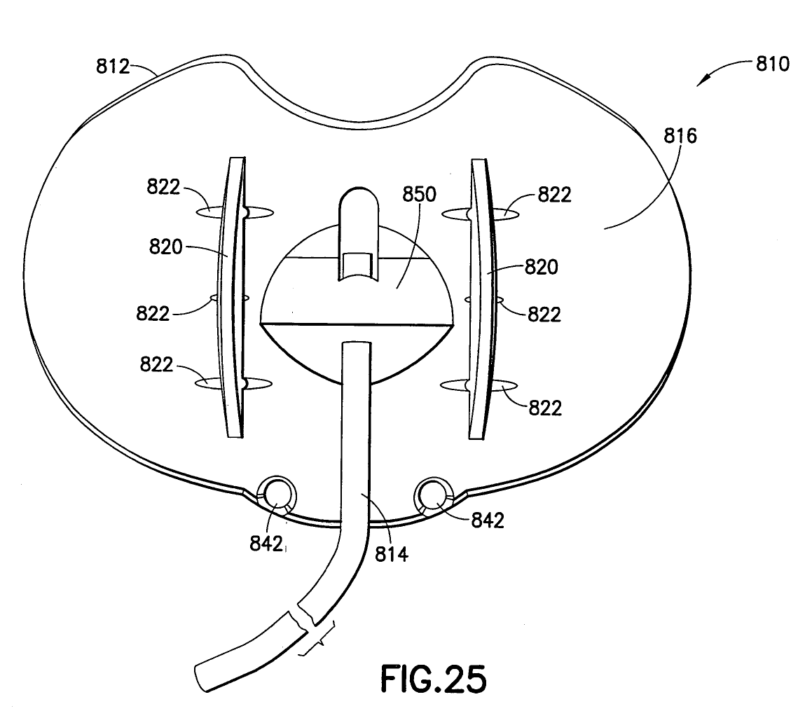

Referring now to FIG. 25, another embodiment of a glaucoma shunt implant 810 according to the invention is shown. The implant 810 includes ribs 820 on the upper surface 816 of the plate 812 extending in an anterior-posterior direction along the plate. Transverse bores 822 are provided through preferably at least a portion of the ribs 820 and optionally a portion of the plate to permit aqueous flow therethrough and potentially tissue ingrowth. Suture holes are provided at an anterior region of the plate. Other aspects of the shunt are similar to previously described shunts (e.g., recessed drainage tube 814 and ridge over the tube 850).

There have been described and illustrated herein embodiments of a glaucoma shunt and method of implanting the same. While particular embodiments of the invention have been described, it is not intended that the invention be limited thereto, as it is intended that the invention be as broad in scope as the art will allow and that the specification be read likewise. Thus, while silicone is a preferred material, it will be appreciated that other preferably flexible materials, including gellans and hydromers may be used as well. In addition, while particular shapes of the implant have been disclosed, it will be understood that the shunt can be formed with other suitable shapes that will not negatively impinge anatomical features. Also, while dimensions and surface areas of preferred embodiments have been disclosed, the invention is not limited thereto. With respect thereto, those embodiments indicated to preferably have a surface area of approximately (or at least approximately) 350 mm², may be smaller in size and have a surface area of 200-350 mm². In addition, in each of the embodiments, the flexible drainage tube may be valved, non-valved and permanently open, or provided with a dissolvable or otherwise removable plug which initially obstructs passage of aqueous through the tube and is later removed (by dissolution, physician action, or other means) to allow increased flow of aqueous through the tube. In addition, each of the embodiments may be provided rimmed with a wall or non-rimmed, and with ribs for lifting the tissue (as shown) or without ribs. It will therefore be appreciated by those skilled in the art that yet other modifications could be made to the provided invention without deviating from scope as claimed.

1. An implant for draining aqueous from an eye, comprising:
a thin, elastomeric plate having first and second surfaces, said first surface curved to spherically to conform to a curvature of the eye, said first surface defining a periphery and a central portion, and a longitudinal recess formed in said plate extending from an anterior portion of said plate to said central portion; and
a drainage tube for draining aqueous from the eye and onto said plate, said tube extending at least partially within said recess and having first and second ends, said first end for insertion into the eye and said second end located in said recess.

2. An implant according to claim 1, wherein:
said second end is located at said central portion of said plate.

3. An implant according to claim 1, further comprising:
a ridge extending upward from said first surface at said central portion, and said tubing extends under or through said ridge.

4. An implant according to claim 1, further comprising:
at least one rib extending along one of said first and second surfaces in an anterior-posterior direction.

5. An implant according to claim 4, wherein:
said at least one rib is on said first surface.

6. An implant according to claim 4, wherein:
said at least one rib is on said second surface.

7. An implant according to claim 4, wherein:
at least one bore is provided through or under said rib.

8. An implant for draining aqueous fluid from an eye, comprising:
a thin, elastomeric plate having first and second surfaces, said first surface curved to spherically to conform to a curvature of the eye, and at least one of said first and second surfaces having anterior-posterior ribs formed thereon; and
a drainage tube having first and second ends for draining aqueous from the eye and onto said plate, said first end for insertion into the eye and said second end located to drain aqueous onto said plate.

9. An implant according to claim 8, wherein:
said ribs are provided on said first surface.

10. An implant according to claim 8, wherein:
said ribs are provided on said second surface.

11. An implant according to claim 8, wherein:
said ribs are provided with transverse bores.

12. An implant according to claim 8, wherein:
said plate has a central portion, and said second end of said tubing is located to empty aqueous at said central portion of said plate.

13. An implant according to claim 12, further comprising:
a ridge extending upward from said first surface at said central portion, and said tubing extends under or through said ridge.

14. An implant for draining aqueous from an eye, comprising:
a single thin, elastomeric plate having first and second surfaces, said first surface defining a periphery and a central portion and curved to spherically to conform to a curvature of the eye; and
a drainage tube having first and second ends for draining aqueous from the eye and onto said plate, said first end for insertion into the eye and said second end located at said central portion of said first surface.

15. An implant according to claim 14, further comprising:
a ridge extending upward from said first surface at said central portion, and said tubing extends under or through said ridge.

16. An implant according to claim 14, wherein:
said plate is longer in an anterior-posterior direction than in a lateral direction transverse to said anterior-posterior direction.

17. An implant for draining aqueous from an eye, comprising:
a thin, elastomeric plate having first and second surfaces, said first surface curved to spherically to conform to a curvature of the eye, said plate defining a posterior filtration portion and an anterior fixation portion, said fixation portion partly defined by a narrow waist at a junction of the filtration and fixation portions; and
a drainage tube having first and second ends, said first end for insertion into the eye and said second end located to empty aqueous onto said plate.

18. An implant according to claim 17, wherein:
a length of the fixation portion from an anterior edge of the fixation portion to an anterior edge of said filtration portion is approximately 4 mm.

19. An implant according to claim 17, wherein:
a length of the fixation portion from an anterior edge of the fixation portion to an anterior edge of said filtration portion is at least 4 mm.

20. An implant for draining aqueous from an eye, comprising:
a thin, elastomeric plate having first and second surfaces, said first surface curved to spherically to conform to a curvature of the eye;
first lateral extensions coupled to said plate with live hinges; and
a drainage tube having first and second ends for draining aqueous from the eye to the plate, said first end for insertion into the eye and said second end coupled relative to said plate.

21. An implant according to claim 20, wherein:
said live hinges are provided at an anterior portion of said plate.

22. An implant according to claim 20, wherein:
said live hinges are provided substantially central along said plate.

23. An implant according to claim 20, wherein:
said plate includes second lateral extensions which extend from said plate.

24. An implant for draining aqueous from an eye, comprising:
a thin, elastomeric plate having first and second surfaces each defining peripheral and central portions, said first surface curved to spherically to conform to a curvature of the eye;
at least one crossbeam extending laterally across a central portion of one of said first and second surfaces; and
a drainage tube having first and second ends for draining aqueous from the eye to the plate, said first end for insertion into the eye and said second end provided adjacent said at least one crossbeam.

25. An implant according to claim 24, wherein:
said at least one crossbeam is provided on said first surface.

26. An implant according to claim 25, further comprising:
a wall about said peripheral portion of said first surface.

27. An implant according to claim 24, wherein:
said at least one crossbeam includes two crossbeams, a channel is defined between said two crossbeams, and said second end of said tube is provided in said channel.

28. An implant according to claim 24, wherein:
said crossbeam is perforate.

29. An implant according to claim 24, further comprising:
an elastomeric flap situated over said at least one crossbeam such that said second end of said drainage tube is covered.
